(19) United States
(12) Patent Application Publication
(10) Pub. No.: US 2024/0366381 A1
(43) Pub. Date: Nov. 7, 2024

(54) TRANSCATHETER ANNULOPLASTY LOCKING SYSTEMS, DEVICES AND METHODS

(71) Applicant: Medtronic, Inc., Minneapolis, MN (US)

(72) Inventors: Yogesh A. Darekar, Irvine, CA (US); Victoria T. Tien, Apple Valley, MN (US); Robyn H. Lawrence, Aliso Viejo, CA (US); Amal Elgamil, Aliso Viejo, CA (US); Claudia See, Irvine, CA (US)

(21) Appl. No.: 18/575,846

(22) PCT Filed: Jun. 29, 2022
(86) PCT No.: PCT/IB2022/056067

§ 371 Date: Dec. 31, 2023

Related U.S. Application Data
(60) Provisional application No. 63/217,580, filed on Jul. 1, 2021.

(51) Int. Cl. A61F 2/24

(52) U.S. Cl. CPC A61F 2/2466; A61F 2/2445; A61F 2220/0016; A61F 2250/001

(57) ABSTRACT

Aspects of the disclosure include systems and devices for delivering an implant to a bodily annulus, such as an annuloplasty implant to a heart valve annulus. Various implants of the disclosure include one or more cinching lines extending through respective flexible tubular members, which are anchored to the annulus. The cinching lines can be tensioned to reshape the annulus. Systems further include least one lock to maintain the position of the cinching lines. Methods of reshaping the implant and locking a position of the reshaped implant are also disclosed.

### FIELD

The present technology is generally related to delivery systems, annuloplasty devices and methods of reducing and/or reshaping a bodily annulus, such as a valve annulus for treatment of mitral regurgitation, for example.

### BACKGROUND

Generally, the anatomy and physiology of the human heart is well known. Of the four one-way valves in the heart, the two inlet valves are the mitral valve of the left side of the heart, and the tricuspid valve on the right side of the heart. The tricuspid valve is located between the right atrium and the right ventricle. The three leaflets of the tricuspid valve laterally terminate at the tricuspid annulus. Blood flows from the superior and inferior vena cava into the right atrium, then through the tricuspid valve during diastole to fill the right ventricle. During ventricular systole, the tricuspid valve is closed and blood is ejected through the pulmonary valve into the pulmonary artery and hence through the lungs. At the end of ventricular systole the pulmonary valve closes. Leaving the lungs, the now oxygenated blood flows into the left atrium and hence through the mitral valve into the left ventricle during ventricular diastole. Finally, at ventricular systole the mitral valve closes and blood is ejected through the aortic valve into the aorta. However, should the mitral valve become regurgitant due to disease then some percentage of the left ventricular stroke volume will flow backwards through the mitral valve into the left atrium. This regurgitation causes the left atrial pressure to rise, in turn causing pulmonary artery pressure to rise, which is reflected back to the right ventricular pressure.

Typically, to treat a patient with functional mitral regurgitation, a physician places an annuloplasty ring on the mitral annulus to reduce the circumference and septal-lateral diameter of the annulus. In degenerative mitral regurgitation patients, annuloplasty rings are utilize to stabilize the mitral annulus, not reduce the annular circumference.

The present disclosure addresses problems and limitations associated with the related art.

### SUMMARY

The techniques of this disclosure generally relate to systems and methods of reducing and/or reshaping a bodily annulus for treatment of valve regurgitation, for example. Aspects of the disclosure provide delivery systems that can cinch an implant (e.g., annuloplasty ring) to reshape a valve annulus, such as a mitral valve annulus, and maintain the position of the implant.

In one aspect, the present disclosure provides a system including a tubular first member defining a first passageway extending along its length extending from a first end of the first member to a second end of the first member. The system also includes a first cinching line extending through the first passageway. In some examples, the first cinching line extends out of the first and second ends of the first member. A tubular second member is also provided and defines a second passageway extending along its length extending from a first end of the second member to a second end of the second member. A second cinching line extends through the second passageway. In some examples, the second cinching line extends out of the first and second ends of the second member. The system further includes a first lock connecting the first end of the first cinching line to the first end of the second cinching line, the first lock including a locked arrangement and an unlocked arrangement. The system also includes a second lock connecting the second end of the second cinching line to the second end of the second cinching line: the second lock including a locked arrangement and an unlocked arrangement.

In another aspect, the present disclosure provides a system including a catheter having a first lumen terminating at a distal end of the catheter. The system includes a tubular first member defining a first passageway extending along its length extending from a first end of the first member to a second end of the first member and a first cinching line extending through the first passageway and out of the first and second ends of the first member. The system also includes a first lock maintained in the first lumen. The first lock including a first tab extending from a first body and the first tab and the first body collectively forming a first opening through which the first line is threaded. The first tab is biased away the first body so that the first tab clamps onto the first cinching line. The first tab can be compressed toward the first body to release clamping the first cinching line. The first lumen is sized to compress the first tab such that the first lock is in an open configuration and the first lock is configured to automatically transition to a closed configuration when the first lock is advanced out of the distal end.

In yet another aspect, the disclosure provides a system including a tubular first member defining a first passageway extending along its length extending from a first end of the first member to a second end of the first member and a cinching line extending through the first passageway. The system additionally includes a tubular second member defining a second passageway extending along its length extending from a first end of the second member to a second end of the second member and the cinching line extending through the second passageway. Additionally, the system includes a first lock at a portion of the cinching line between the second end of the tubular first member and the first end of the tubular second member, the first lock including a locked arrangement and an unlocked arrangement.

In another aspect, the disclosure provides a method of repairing or resizing an annulus, the method including providing a delivery device including a catheter defining a first lumen that terminates at a distal end of the catheter. The method further including anchoring a first member around the annulus: wherein the first cinching line extends through a first passageway of the first member between first and second ends of the first member. The first cinching line extends through an opening in a first lock that is positioned within the first lumen. The method also includes distally advancing the first lock out of the first lumen such that the first lock automatically transitions from an open arrangement to a closed arrangement.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIGS. 1-7 schematically illustrate a system implanting an implant around a heart valve annulus.

FIG. 8A is a cross-sectional illustration a lock of the system of FIGS. 1-7 in an unlocked delivery arrangement, positioned within a catheter.

FIG. 8B is a cross-sectional illustration a lock of the system of FIGS. 1-7 in a locked arrangement, positioned outside of the catheter.

FIG. 9A is a perspective view of the lock of FIGS. 8A-8B.

FIG. 9B is a cross-sectional view of the lock of FIGS. 8A-9A.

FIG. 10A is a side view of the lock of FIGS. 8A-9B in the locked arrangement.

FIG. 10B is a side view of the lock of FIGS. 8A-10A in the unlocked arrangement.

FIG. 11A is a perspective view of an alternate lock that can be used with systems of the disclosure.

FIG. 11B is a side view of the alternate lock of FIG. 11A that can be used with systems of the disclosure engaged with a cinching line.

FIG. 12 is a cross-sectional view of a handle assembly that can be used to secure and position the lock of FIGS. 11A-11B with the cinching line.

FIGS. 13A-13B are side views of a member of that can be used with an implant, such as the implant of FIGS. 1-7.

FIGS. 14A-14B schematically illustrate an anchor that can be used with the system of FIGS. 2-7.

FIGS. 15A-15B schematically illustrate another anchor that can be used with the system of FIGS. 1-7.

FIGS. 16A-16B schematically illustrate yet another an anchor that can be used with the system of FIGS. 1-7.

FIGS. 17A-17B schematically illustrate an alternate anchor that can be used with the system of FIGS. 1-7.

FIGS. 18A-1B schematically illustrate an alternate anchor that can be used with the system of FIGS. 1-7.

FIGS. 19A-19B schematically illustrate yet another anchor that can be used with the system of FIGS. 1-7.

FIG. 20 is a schematic illustration of an alternate implant that can be incorporated into systems of the disclosure.

### DETAILED DESCRIPTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

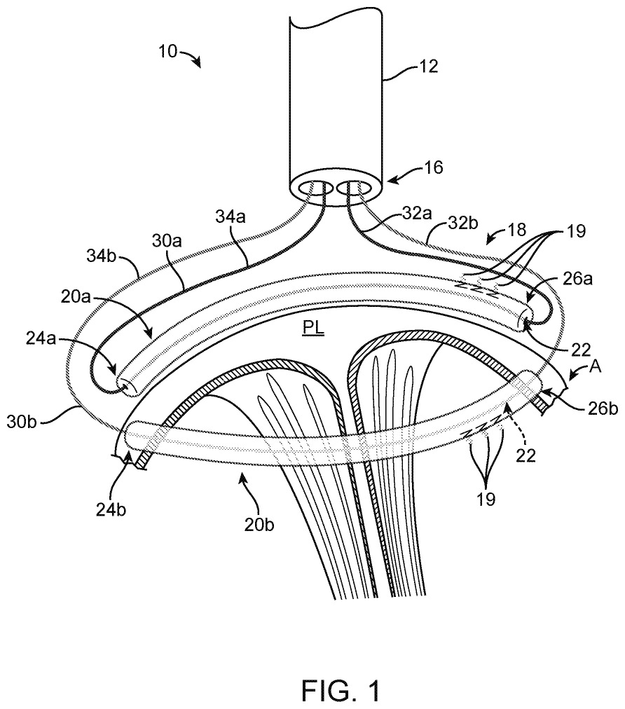

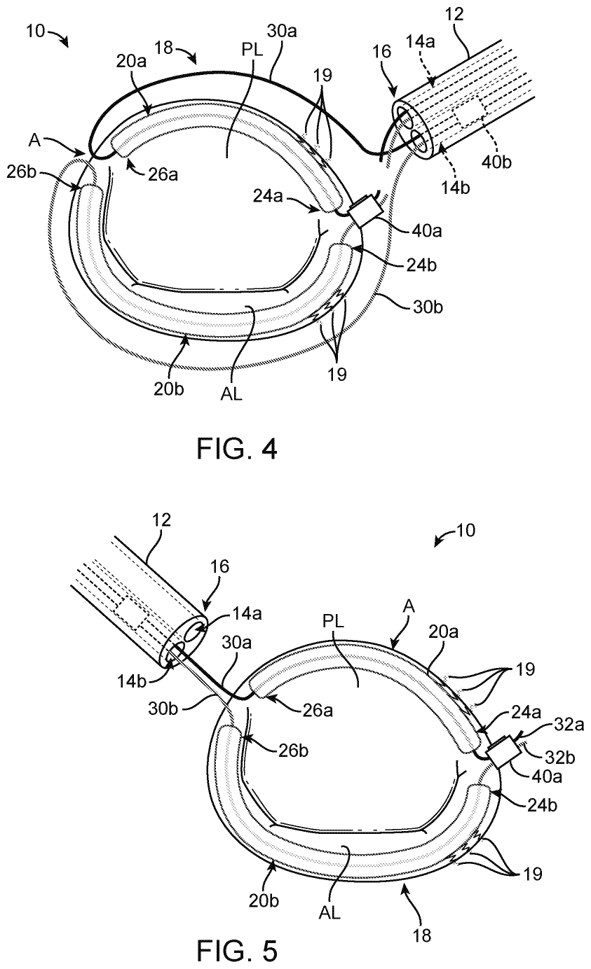

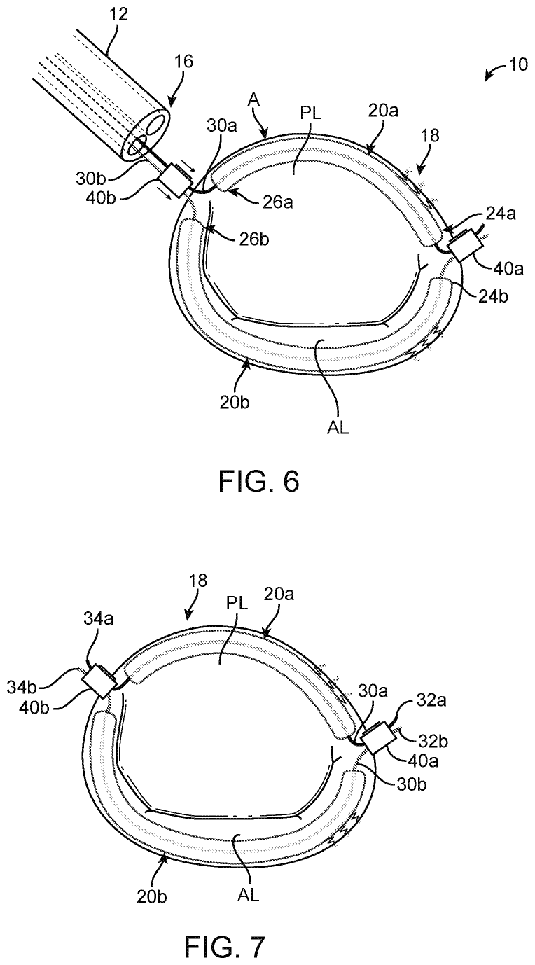

FIGS. 1-7 illustrate a system 10 of the disclosure including a catheter 12 forming a first lumen 14a and a second lumen 14b. Each lumen 14a, 14b terminates at a distal end 16 of the catheter 12. The system 10 further includes an implant 18 secured to a valve annulus A to reshape the annulus. For example, the implant 18 can be secured with a plurality of tissue anchors 19 of any of the types disclosed herein. In the illustrated example, the annulus A is a mitral valve annulus having an anterior leaflet AL and a posterior leaflet PL. It is to be understood that the systems and techniques of the disclosure can be applied to another heart valve or bodily annulus in a similar manner. In this example, the implant 18 includes a first member 20a defining a passageway 22a extending along its length extending from a first end 24a to a second end 26a. At least one cinching line 30a extends through the passageway 22a from the first end 24a to the second end 24b of one respective member 20a. Each cinching line 30a, 30b has a first end 32a, 32b and a second end 34a, 34b and can be a suture, wire, filament or the like. The ends 32a, 32b, 43a, 34b are portions of the respective cinching line 30a, 30b relative to the ends 24a, 24b, 26a, 26b of the members 20a, 20b. In one example, the first member 20a can be configured for securing along the posterior leaflet PL. A second member 20b can further be provided and can optionally be identically configured to the first member 20a. The second member 20b can define a passageway 22b extending along its length extending from a first end 24b to a second end 26b. At least one cinching line 30b extending through the passageway 22b from the first end 24b to the second end 26b. In one example, the second member 20b can be configured for securing along the anterior leaflet AL. In various embodiments, the first and/or second members 20a, 20b can be C-shaped or D-shaped prior to and/or after cinching or can be formed of multiple angled segments. The first ends 32a, 32b of the respective cinching lines 30a, 30b are adjacent and the second ends 34a, 34b of the respective cinching lines 30a, 30b are adjacent. Tension can be applied to one or more the ends 32a, 32b, 34a, 34b of the cinching lines 30a, 30b to reshape the annulus A as desired.

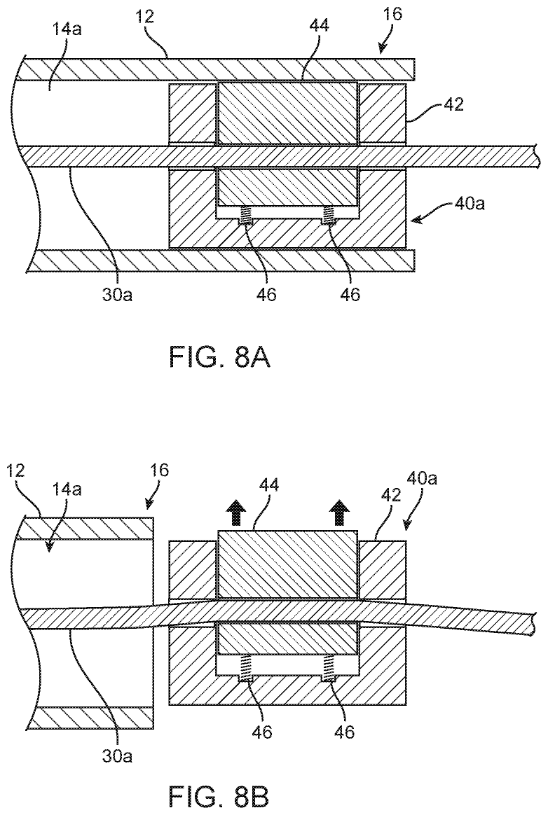

Referring now in addition to FIGS. 8A-10B, once the desired tensioning or cinching of the cinching lines 30a, 30b resulting in reshaping of at least one of the first and second members 20a, 20b (and the annulus A secured thereto) has been achieved, locks 40a, 40b can be positioned on the cinching lines 30a, 30b proximate each end of the members 20a, 20b to maintain the position of the implant 18 around the annulus A by restricting movement of the first and second ends 32a, 32b, 34a, 34b of the respective cinching lines 30a, 30b to a longitudinal position within the respective passageway 22a, 22b. In one example, each lock 40a, 40b can have a maximum outer diameter greater than a diameter of the respective passageway 22a, 22b so that the lock 40a, 40b cannot fit within the respective passageway 22a, 22b.

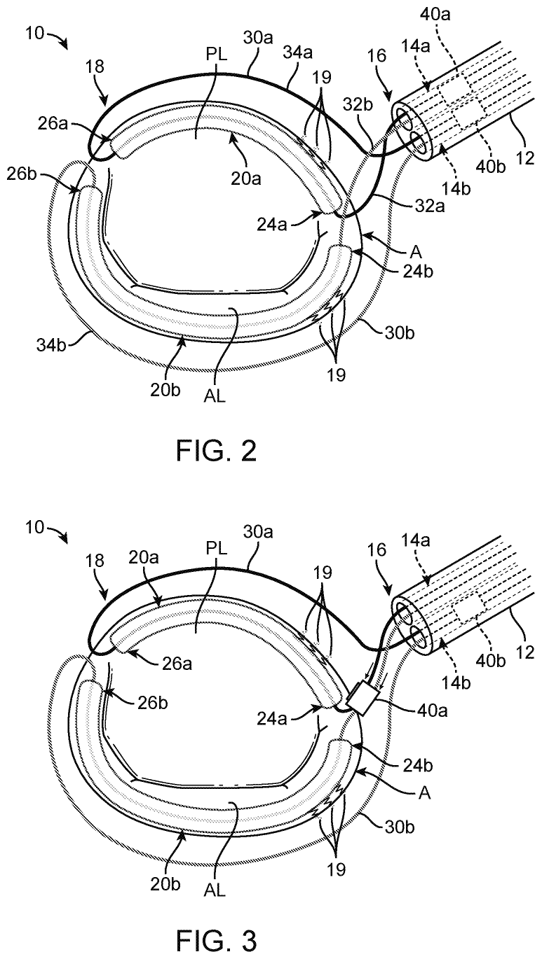

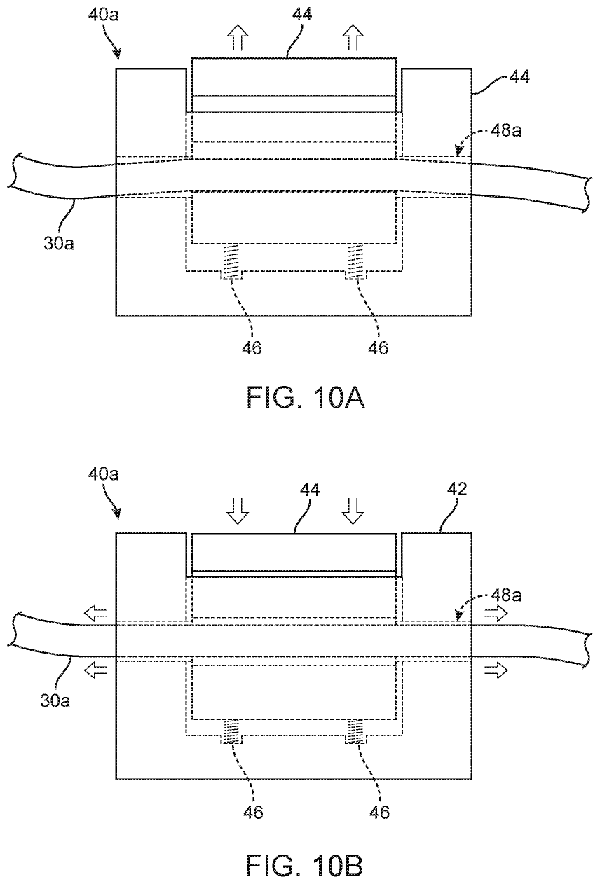

As is perhaps best shown in FIGS. 2 and 8A-8B, in one example, two opposing locks 40a, 40b are provided in a delivery arrangement within respective lumens 14a, 14b of the catheter 12 in which the locks 40a, 40b are forced into the unlocked arrangement due to the size of the respective lumen 14a, 14b. Each lock 40a, 40b of the disclosure can optionally be identically configured. Therefore, only one lock 40a is shown and described in detail in FIGS. 8A-10B. In this example, each lock 40a includes a body 42 and a tab 44 that is biased away from the body 42 with one or more springs 46 or the like. The body 42 and tab 44 collectively form at least one opening (e.g., openings 48a, 48b) through which one or more cinching lines 30a, 30b can be routed. In the illustrated example, the body 42 and tab 44 collectively form two parallel openings 48a, 48b, one opening for each cinching line 30a, 30b. When the tab 44 is compressed (FIG. 10B), the lock 40a is in an unlocked arrangement in which the cinching lines 30a, 30b are free to move through the opening(s) 48a, 48b. When the tab 44 is released (FIG. 10A), the tab 44 transitions to its natural, locked arrangement, biased away from the body 42, such that the cinching line(s) 30a, 30b are compressed by the tab 44 and body 42, restricted from movement through the respective opening(s) 48a, 48b. In various examples of the disclosure, each lock 40a, 40b is delivered to the annulus A within one respective lumen 14a, 14b of the catheter 12. Each lumen 14a, 14b is sized such that when the respective lock 40a, 40b is within the lumen, the tab 44 will be compressed to a degree where the lock 40a, 40b is in the unlocked arrangement. When the lock 40a, 40b is distally advanced out of and freed from the confines of the respective lumen 14a, 14b, the biased nature of the tab 44 will automatically transition the tab 44 to the locked arrangement to effectively lock, via compressional force, any cinching lines 30a, 30b routed through the lock 40a, 40b in position so that the cinching lines 30a, 30b cannot move though the opening(s) 48a, 48b. Each lock 40a, 40b can be distally advanced in any of a number of ways such as pushing the lock out of the respective lumen 14a, 14b, for example.

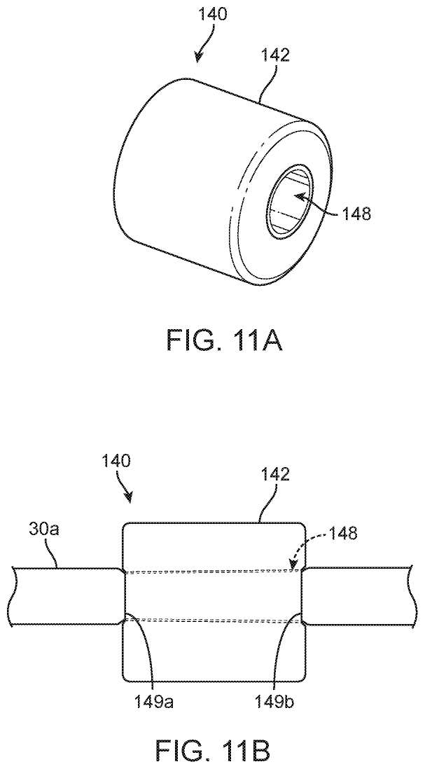

Referring now in addition to FIGS. 11A-11B, which illustrate an alternate lock 140 of the disclosure, which can be used as a replacement for one or more of the locks 40a, 40b. In this embodiment, each lock 140 includes a body 142 defining an opening 148 having a first end 149a and a second end 149b. In one example, the first end 149a has a smaller diameter as compared to the second end 149b. In various examples, the opening 148 tapers from the second end 149b to the first end 149a. The body 142 can take a variety of shapes and is sized such that at least a portion of the body 142 is larger than and cannot fit within the passageway of an adjacent member (e.g., members 20a, 20b). The opening 148 is sized such that one or more cinching lines (e.g., 30a, 30b) can be forced to slide through the opening 148 by pulling the cinching line (i.e. to put the lock 140 in position adjacent one end of the first or second member) but will frictionally maintained in position within the opening 148 when the pulling force is removed.

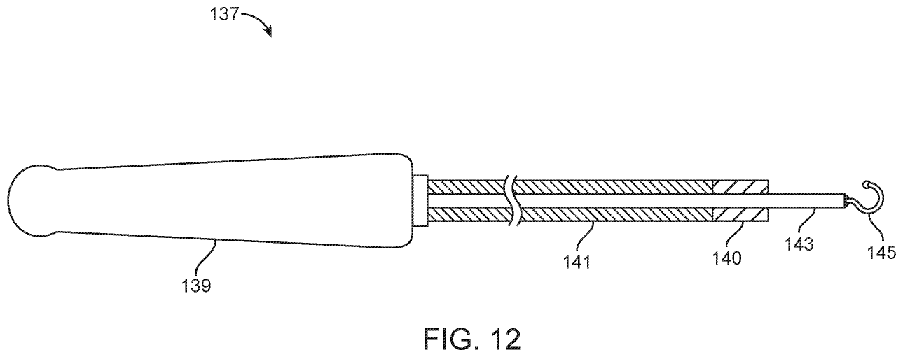

The lock 140 of FIGS. 11A-11B can optionally be loaded onto one or more cinching lines 30a, 30b with a handle assembly 137 as is shown in FIG. 12. In various embodiments, the handle assembly 137 includes a handle 139 configured to be handled by a clinician and remain outside of the patient, a push rod 141, a pull rod 143 coaxially slidable within the push rod 141 and a hook 145 secured to a distal end of the pull rod 143. The first end 149a of the opening 148 (having a small diameter) is oriented toward the handle 139. Then, the lock 140 is slid over the pull rod 143 and positioned adjacent the push rod 141 as is shown in FIG. 12. This step can be performed outside the patient body. Additional steps are performed inside the patient body following standard access site operations are completed with introducer sheath (not shown) and access catheter (e.g., catheter 12). The lock 140, being loaded onto the handle assembly 137, is inserted into the catheter lumen (e.g., a lumen in catheter 12) to maneuver the lock 140 to the left atrium location, for example. The lock 140 loaded onto the handle assembly 137 is maneuvered to the desired location with adjusting the distal end 16 of the catheter 12 to grab or hook 145 one or more cinching lines 30a, 30b. To confirm the cinching line(s) 30a, 30b is engaged, the handle assembly 137 is tugged proximally and by experiencing the load confirm the cinching line (between first member 20a and second member 20b) is grabbed by the hook 145. Once confirmed, the push rod 141 is used to slide the second end 149b of the lock 140 distally along the pull rod 143 and finally onto the cinching line 30a, 30b. Then, proximally pull the cinching line 30a, 30b further to move the lock 140 toward the first or second member 20a, 20b direction and cinching line 30a, 30b proximally towards the catheter 12 and thereby reducing the annulus and finally reducing annulus, which can reduce mitral regurgitation in the case if a mitral valve annulus.

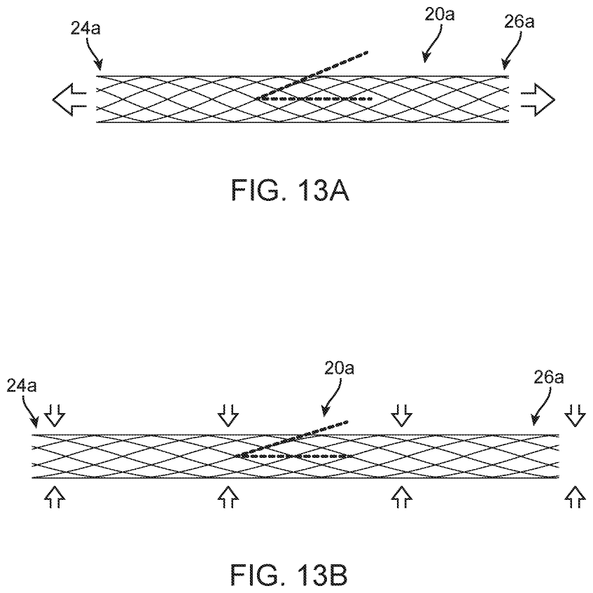

The first and second members 20a, 20b can be made of a flexible material. In some examples, the first and second members 20a, 20b can be described as tubular forming the passageway 22a, 22b with first and second ends 24a, 24b, 26a, 26b. In various examples, the first and/or second members 20a, 20b can be made of a metal mesh such as, but not limited to, helical mesh or biaxial mesh. One example of such biaxial mesh is seen in FIGS. 13A-13B. In this example, it can be shown that when first and second ends 24a, 24b of the first member 20a are pulled in opposite directions, an outer diameter of the first member 20a decreases along a length of the first member 20a. It will be understood that if the second member 20b is made of a similar material, the second member 20b will behave in a similar way.

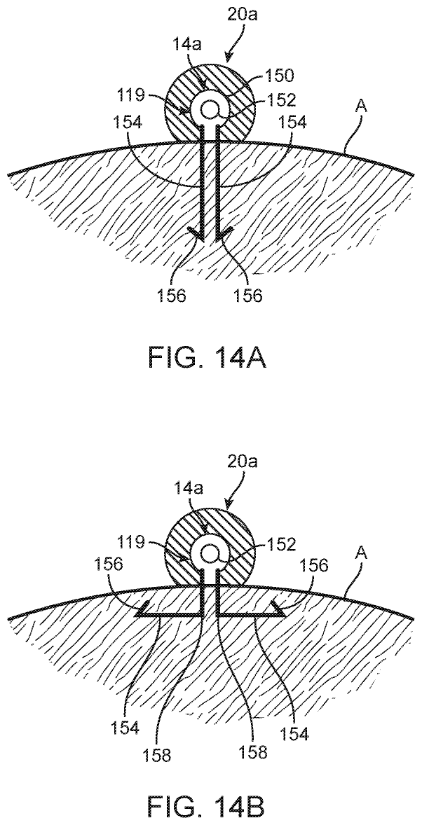

As indicated above, systems of the disclosure can include a plurality of tissue anchors 19 provided to secure the first or second members 20a, 20b to tissue forming the annulus A. Such anchors 19 can be delivered and deployed via any known method. One example of such an anchor is shown in FIGS. 14A-14B. In this example, an anchor 119 can include a ring 150 defining an opening 152. Extending from the ring 150 can be one or more prongs 154, each terminating at one or more barbs 156 angled toward the ring 150. The anchor 119 can be inserted into tissue in a delivery arrangement (FIG. 14A) and then the anchor 119 can be configured to automatically transition to a deployed arrangement (FIG. 14B) once inserted into the annulus A. In one example, each prong 154 can be made of a shape memory material biased to assume the configuration of FIG. 14B. In the illustrated example, each prong 154 may be biased to form a bend or angle 158.

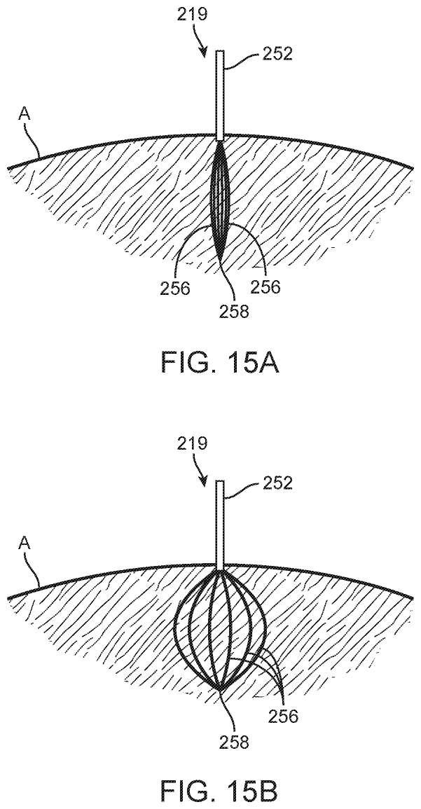

Yet another anchor 219 is shown in FIGS. 15A-15B. In this example, one or more anchors can include a prong 252 terminating at a plurality of barbs 256 (Generally referenced). In the illustrated example, the plurality of barbs 256 can be delivered in a compressed or elongated, delivery arrangement (FIG. 15A). Once inserted into the tissue forming the annulus A, the barbs 256 can transition to a deployed arrangement (FIG. 15B) to bend away from a central axis of the prong 252 collectively form a generally spherical or ovoid shape, converging at both a distal end of the prong 252 and at a distal tip 258. In one example, the barbs 256 are made of a shape memory material and biased to the position of FIG. 15B.

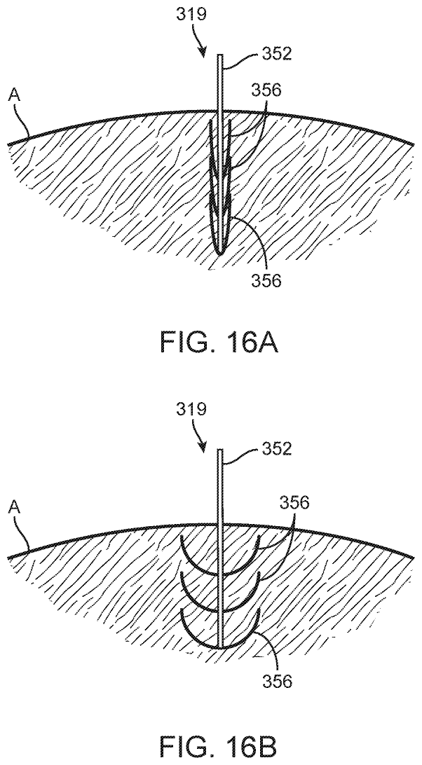

Another alternate barb 319 is illustrated in FIGS. 16A-16B. In this example, the barb 319 includes a prong 352 having a plurality of barbs 356. Each barb 356 can be arcuate or generally U-shaped, connected to the prong 352 at an approximate longitudinal midsection of the barb 356. In the illustrated example, the plurality of barbs 356 can be delivered in a delivery arrangement (FIG. 16A), compressed against or toward the prong 354. Once inserted into the tissue forming the annulus A, the barbs 356 can transition to a deployed arrangement (FIG. 16B) to extend away from the prong 352. In one example, the barbs 356 are made of a shape memory material and biased to the position of FIG. 16B.

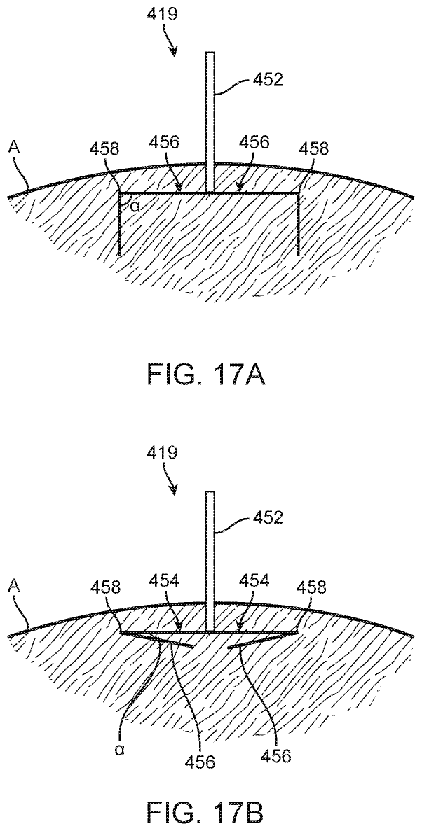

Another alternate barb 419 is illustrated in FIGS. 17A-17B. In this example, the barb 419 includes a prong 452 having a plurality of barbs 456. Each barb 456 can include a bend or angled portion 458. In the illustrated example, the plurality of barbs 456 can be delivered in a delivery arrangement (FIG. 17A. Once inserted into the tissue forming the annulus A, the barbs 456 can transition to a deployed arrangement (FIG. 17B) to decrease an angle α formed by the angled portion 458. In one example, the barbs 456 are made of a shape memory material and biased to the position of FIG. 17B.

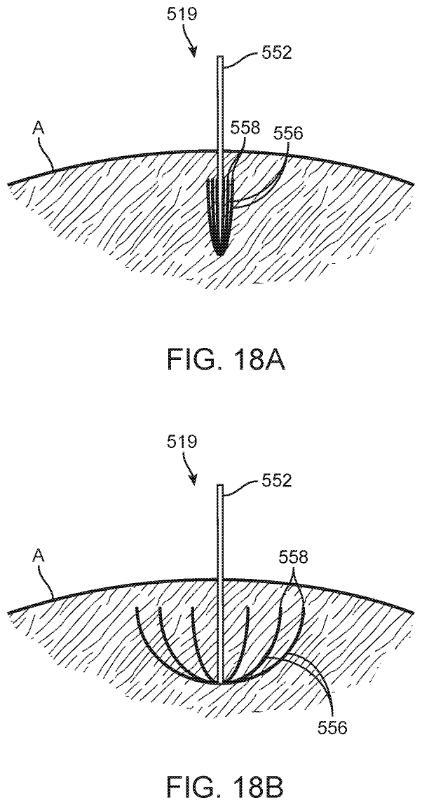

Yet another example of a suitable anchor 519 is shown in FIGS. 18A-18B. In this example, one or more anchors can include a prong 552 terminating at a plurality of barbs 556 (generally referenced). In the illustrated example, the plurality of barbs 556 can be delivered in a compressed, delivery arrangement (FIG. 18A) generally positioned against prong 552. Once inserted into the tissue forming the annulus A, the barbs 556 can transition to a deployed arrangement (FIG. 18B) to bend away from a central axis of the prong 552. In the deployed arrangement, ends 558 (only a select few are referenced) extend toward a proximal end of the prong 552 so that the anchor 519 generally forms the shape of an umbrella. In one example, the barbs 556 are made of a shape memory material and biased to the position of FIG. 18B.

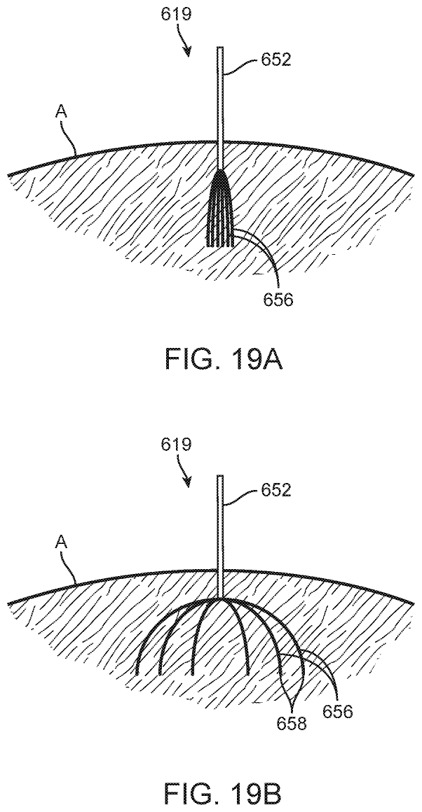

Another example of a suitable anchor 619 is shown in FIGS. 19A-19B. In this example, one or more anchors can include a prong 652 terminating at a plurality of barbs 656 (generally referenced). In the illustrated example, the plurality of barbs 656 can be delivered in a compressed, delivery arrangement (FIG. 19A) generally positioned against prong 652. Once inserted into the tissue forming the annulus A, the barbs 656 can transition to a deployed arrangement (FIG. 19B) to bend away from a central axis of the prong 652. In the deployed arrangement, ends 658 (only a select few are referenced) extend away from a distal end of the prong 652. In one example, the barbs 656 are made of a shape memory material and biased to the position of FIG. 19B.

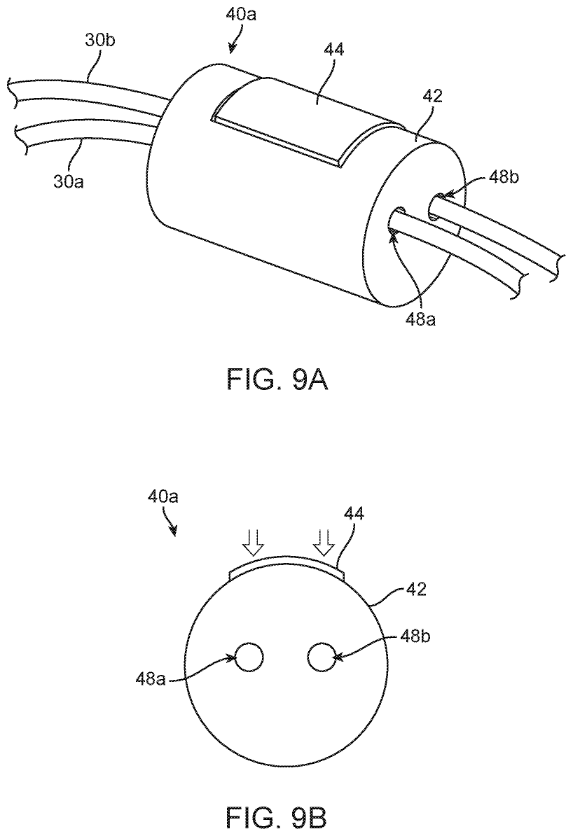

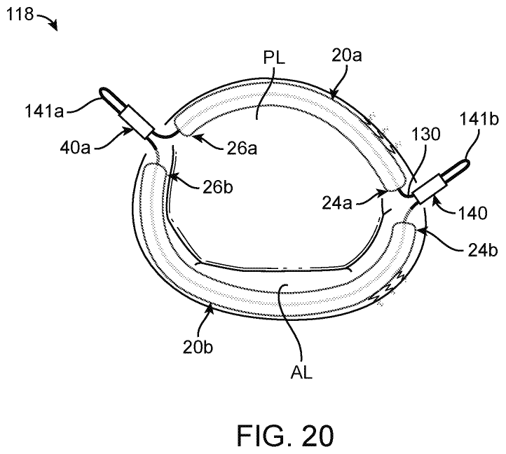

Referring in addition to FIG. 20, which illustrates an alternate implant 118 that can be incorporated into the systems of the disclosure. As indicated with like reference numbers, the implant 118 includes many elements previously disclosed. The main difference between implant 118 and those previously disclosed is that a single cinching line 130 is threaded or looped through first and second members 20a, 20b. The cinching line 130 can be of any of the type disclosed herein. In this example, the cinching line 130 forms a loop 141a one end 26a, 26b of the first and second members 20a, 20b and a second loop 141b at the opposing end 24a, 24b of the first and second members 20a, 20b. One lock (e.g., lock 40a) can be secured at the first loop 141a to maintain the cinching line 130 at the respective end 26a, 26b and second lock (e.g., lock 140) can be secured at the second loop 141b to maintain the cinching line 130 at the opposing end 24a, 24b of the first and second members 20a, 20b. It could be interpreted that the locks 40a, 140 form part of the respective loops 141a, 141b by closing the respective loop and maintaining a length of the cinching line 130 forming the respective loop. In various embodiments, the lock 40a may only a single opening 48a (see also, FIG. 9A). It will be understood that locks 40a and 140, or any alternate lock disclosed herein that is utilized with the implant 118, can optionally be delivered and deployed in a manner similar to that disclosed above with respect to the particular lock design. The present disclosure is intended to include all variations in which a single cinching line 130 interconnects two members 20a, 20b and can be secured around an annulus in a cinched arrangement in which the space between the first and second members 20a, 20b is adjusted and locked into position with at least one lock.

Various methods of the disclosure can include repairing or resizing an annulus. Various methods including providing a delivery device including a catheter defining a first lumen that terminates at a distal end of the catheter. The method further including anchoring a first member around the annulus: wherein the first cinching line extends through a first passageway of the first member between first and second ends of the first member. The first cinching line extends through an opening in a first lock that is positioned within the first lumen. The method also includes distally advancing the first lock out of the first lumen such that the first lock automatically transitions from an open arrangement to a closed arrangement. Various methods include the step of severing the first cinching line adjacent each of the first lock. In some examples, the first member is made of a mesh having a configuration that is selected from the group consisting of helical mesh or biaxial mesh. Various methods include the step of tensioning at least one of the first cinching line to reshape the annulus prior to distally advancing the lock out of the first lumen. In some embodiments, the annulus is a heart valve annulus. Some methods include anchoring a second member around the annulus: wherein a second cinching line extends within a second passageway of the second member from a first end of the second member to a second end of the second member. In various examples, the first end of the second cinching line extends through the first lock and the first and second cinching lines extend through a second lock positioned adjacent the second ends of the first and second members. In methods relating to the implant of FIG. 20, one cinching line extends through the first and second lock twice to form loops adjacent the ends of the first and second members to secure the implant in a cinched arrangement around an annulus. Some methods include the step of distally advancing the second lock out of a second lumen of the catheter such that the second lock automatically transitions from an open arrangement to a closed arrangement.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity: it should be understood that the techniques of this disclosure may be performed by a combination of units or modules associated with, for example, a medical device.

1. A system comprising:
a tubular first member defining a first passageway extending along its length extending from a first end of the first member to a second end of the first member;
a first cinching line extending through the first passageway;
a tubular second member defining a second passageway extending along its length extending from a first end of the second member to a second end of the second member;
a second cinching line extending through the second passageway;
a first lock connecting a first end of the first cinching line to a first end of the second cinching line, the first lock including a locked arrangement and an unlocked arrangement; and
a second lock connecting the second end of the second cinching line to the second end of the second cinching line; the second lock including a locked arrangement and an unlocked arrangement.

2. The system of claim 1, wherein the first lock has a first tab extending from a first body; the first tab and the first body collectively forming at least one opening through which the first and second cinching lines are threaded; wherein the first tab is biased away the first body so that the first tab clamps onto the first and second cinching lines; wherein the first tab can be compressed toward the first body to release clamping the first and second cinching lines.

3. The system of claim 2, wherein the second lock has a second tab extending from a second body; the second tab and the second body collectively forming at least one opening through which the first and second cinching lines are threaded; wherein the second tab is biased away the second body so that the second tab clamps onto the first and second cinching lines; wherein the second tab can be compressed toward the second body to release clamping of the first and second cinching lines.

4. A system comprising:
a catheter including a first lumen terminating at a distal end of the catheter;
a tubular first member defining a first passageway extending along its length extending from a first end of the first member to a second end of the first member;
a first cinching line extending through the first passageway and out of the first and second ends of the first member; and
a first lock maintained in the first lumen; the first lock including a first tab extending from a first body; the first tab and the first body collectively forming a first opening through which the first line is threaded; wherein the first tab is biased away the first body so that the first tab clamps onto the first cinching line; wherein the first tab can be compressed toward the first body to release clamping the first cinching line;
wherein the first lumen is sized to compress the first tab such that the first lock is in an open configuration; wherein the first lock is configured to automatically transition to a closed configuration when the first lock is advanced out of the distal end.

5. The system of claim 4, wherein the catheter includes a second lumen terminating at the distal end and a tubular second member defining a second passageway extending along its length extending from a first end of the second member to a second end of the second member; a second cinching line extending through the second passageway and out of the first and second ends of the second member; the system further comprising a second lock positioned within the second lumen.

6. The system of claim 5, wherein the second lumen is sized to compress a second tab of the second lock such that the second lock is in an open configuration; wherein the second lock is configured to automatically transition to a closed configuration when the second lock is advanced out of the distal end.

7. The system of claim 5, wherein the second cinching line extends though the first passageway and the first cinching line extends through the second passageway.

8. The system of claim 4, wherein the first member is made of a mesh having a configuration that is selected from the group consisting of helical mesh or biaxial mesh. 9. The system of claim 4, further comprising a plurality of tissue anchors embedded within the first member.

10. A method of resizing an annulus comprising:
providing a delivery device including a catheter defining a first lumen that terminates at a distal end of the catheter;
anchoring a first member around the annulus; wherein the first cinching line extends through a first passageway of the first member between first and second ends of the first member; further wherein the first cinching line extends through an opening in a first lock that is positioned within the first lumen; and
distally advancing the first lock out of the first lumen such that the first lock automatically transitions from an open arrangement to a closed arrangement.

11. The method of claim 10, further comprising the step of severing the first cinching line adjacent each of the first lock.

12. The method of claim 10, further comprising the step of tensioning at least one of the first cinching line to reshape the annulus prior to distally advancing the lock out of the first lumen.

13. The method of claim 10, further comprising anchoring a second member around the annulus; wherein a second cinching line extends within a second passageway of the second member from a first end of the second member to a second end of the second member.

14. The method of claim 13, wherein the first end of the second cinching line extends through the first lock; further wherein the first and second cinching lines extend through a second lock positioned adjacent the second ends of the first and second members.

15. The method of claim 14, further comprising the step of distally advancing the second lock out of a second lumen of the catheter such that the second lock automatically transitions from an open arrangement to a closed arrangement.

16. (canceled)

17. (canceled)

18. The system of claim 1, further comprising a plurality of tissue anchors embedded within the first and second members.

19. (canceled)

20. (canceled)
